(19) United States
(12) Patent Application Publication
(10) Pub. No.: US 2024/0148734 A1
(43) Pub. Date: May 9, 2024

(54) SELECTIVE INHIBITOR OF EXON 20 INSERTION MUTANT EGFR

(71) Applicant: Taiho Pharmaceutical Co., Ltd., Tokyo (JP)

(72) Inventors: Kazutaka Miyadera, Tsukuba-Shi (JP); Yoshimi Aoyagi, Tsukuba-Shi (JP); Shinichi Hasako, Tsukuba-Shi (JP)

(73) Assignee: Taiho Pharmaceutical Co., Ltd., Tokyo (JP)

(21) Appl. No.: 18/512,528

(22) Filed: Nov. 17, 2023

Related U.S. Application Data
(63) Continuation of application No. 16/345,792, filed on Apr. 29, 2019, now Pat. No. 11,857,513.

(30) Foreign Application Priority Data
Oct. 31, 2016 (JP) 2016-213072

(51) Int. Cl. A61K 31/519; A61P 35/00

(52) U.S. Cl. CPC A61K 31/519; A61P 35/00

(57) ABSTRACT

An antitumor agent comprising a compound selected from the group consisting of Compounds A to D described in the specification, or a salt thereof, for treating a malignant tumor patient expressing EGFR having exon 20 insertion mutation.

### CROSS-REFERENCE TO RELATED APPLICATIONS

The present application is a continuation of U.S. Application No. 16/345,792, filed Apr. 29, 2019, which is a 35 U.S.C. § 371 National Phase Application of International Application No. PCT/JP2017/037186, filed Oct. 31, 2017, which is based upon and claims the benefit of priority to Japanese Application No. 2016-213072, filed Oct. 31, 2016. The entire contents of all of the above applications are incorporated herein by reference.

In accordance with 37 CFR § 1.833-1835 and 37 CFR§ 1.77(b)(5) , the specification makes reference to a Sequence Listing submitted electronically as a .xml file named “550608US_ST26.xml”. The .xml file was generated on Nov. 15, 2023 and is 2,925 bytes in size. The entire contents of the Sequence Listing are hereby incorporated by reference.

### Technical Field

The present invention relates to an antitumor agent against cancers, comprising an exon 20 insertion mutant epidermal growth factor receptor (hereinafter referred to as “EGFR”).

### Background Art

EGFR is a receptor-type tyrosine kinase, exerts its physiological function in normal tissue by being bound to Epidermal Growth Factor (hereinafter also referred to as EGF), which is a ligand, and contributes to growth and apoptosis inhibition in epithelial tissues (NPL 1). Further, somatic mutation of EGFR gene has been known as a cancer-causing gene; for example, EGFR in which the 746th to 750th amino acids in the exon 19 region are deleted (hereinafter also referred to as “exon 19 deletion mutation”) and EGFR in which the 858th amino acid in the exon 21 region is mutated from leucine to arginine (hereinafter also referred to as “L858R mutation”) constantly induces EGF-independent kinase activity, and contributes to the growth and survival of cancer cells (NPL 2). These mutations are observed, for example, in 30 to 50% of non-small-cell lung cancer in East Asia. The mutations are also observed in about 10% of non-small-cell lung cancer in Europe and the United States, and is regarded as one of the causes of cancers (NPL 3).

Therefore, research and development of EGFR inhibitor as an antitumor agent have actively been conducted, and introduced into the treatment of EGFR mutation-positive lung cancer. For example, although administration of gefitinib, erlotinib, and afatinib in their therapeutic dose causes, as side effects, digestive tract disorders and skin disorders, which are widely thought to be attributable to inhibition of wild-type EGFR, they exert a high antitumor effect against exon 19 deletion mutant and L858R mutant EGFR-positive lung cancers. The therapeutic effects of these agents are assumed to be derived from selective inhibition against mutant EGFR, compared with wild-type EGFR, by an EGFR inhibitor (NPL 4).

However, recent studies found that some cancers have EGFR with a mutation in which one or more amino acids are inserted in the exon 20 region (hereinafter also referred to as “exon 20 insertion mutation”), and that these cancers have low sensitivity with respect to previously known EGFR inhibitors. For example, there are clinical reports showing significantly lower antitumor effects of afatinib against EGFR mutation-positive lung cancer with respect to exon 20 insertion mutation, compared with exon 19 deletion mutation or L858R mutation (NPL 5). For this reason, chemotherapies have been used for the patients with these cancers. However, since the treatment options are limited and sufficient therapeutic effects have not been obtained, an antitumor agent with further higher therapeutic effects has been demanded.

PTL 1 discloses a compound usable for the treatment of diseases characterized by exon 20 insertion mutant EGFR. However, the compound of PTL 1 greatly differs in its structure from the compound according to the present invention, and PTL 1 nowhere discloses selectivity based on a comparison with wild-type EGFR, or efficacy in an in vivo model.

Further, although PTL 2 discloses a quinoline-substituted compound, PTL 2 nowhere discloses inhibitory activity against exon 20 insertion mutant EGFR.

### CITATION LIST

### Patent Literature

PTL 1: WO2015/175632A1

PTL 2: WO2015/025936A1

### Non-Patent Literature

NPL 1: Nat. Rev. Cancer, Vol. 6, pp. 803-812 (2006)

NPL 2: Nature Medicine, Vol. 19, pp. 1389-1400 (2013)

NPL 3: Nat. Rev. Cancer, Vol. 7, pp. 169-181 (2007)

NPL 4: Lancet Oncol. Vol. 13, e. 23-31 (2012)

NPL 5: Lancet Oncol. Vol. 16, pp. 830-838 (2015)

### SUMMARY OF INVENTION

### Technical Problem

An object of the present invention is to provide an antitumor agent with reduced side effects derived from the inhibition of wild-type EGFR, the antitumor agent serving as an inhibitor that can ensure high selectivity with respect to exon 20 insertion mutant EGFR for which the therapeutic effects of the previously known EGFR inhibitors are insufficient.

### Solution to Problem

The inventors of the present invention conducted extensive research, and found that exon 20 insertion mutant EGFR is an appropriate target in treating cancers, and that EGFR inhibitors conventionally used for the treatments have inferior selectivity between wild-type EGFR and exon 20 insertion mutant EGFR. Further, the inventors also confirmed that a specific compound exerts selectivity with respect to exon 20 insertion mutant EGFR and tumor growth inhibitory effects, and is thus regarded as superior to afatinib, which is a typical EGFR mutation-positive cancer-treating agent. With this finding, the inventors accomplished the present invention.

Accordingly, the present invention encompasses the following embodiments.

### Item 1.

An antitumor agent for treating a malignant tumor patient expressing EGFR having exon 20 insertion mutation, the antitumor agent comprising a compound selected from the group consisting of:
(S)-N-(4-amino-6-methyl-5-(quinolin-3-yl)-8,9-dihydropyrimido[5,4-b]indolizin-8-yl)acrylamide (hereinafter also referred to as Compound A);
(S)-N-(4-amino-6-methylene-5-(quinolin-3-yl)-7,8-dihydro-6H-pyrimido[5,4-b]pyrrolizin-7-yl)acrylamide (hereinafter also referred to as Compound B);
(S,E)-N-(4-amino-6-methylene-5-(quinolin-3-yl)-7,8-dihydro-6H-pyrimido[5,4-b]pyrrolizin-7-yl)-3-chloroacrylamide (hereinafter also referred to as Compound C); and
(R)-N-(4-amino-6-methyl-5-(quinolin-3-yl)-8,9-dihydropyrimido[5,4-b]indolizin-8-yl)-N-methylacrylamide (hereinafter also referred to as Compound D),
or a salt thereof.

### Item 2.

The antitumor agent according to Item 1, wherein the compound is (S)-N-(4-amino-6-methyl-5-(quinolin-3-yl)-8,9-dihydropyrimido[5,4-b]indolizin-8-yl)acrylamide.

### Item 3.

The antitumor agent according to Item 1 or 2, wherein the malignant tumor patient expressing EGFR having exon 20 insertion mutation is a patient with lung cancer, breast cancer, head and neck cancer, brain tumor, uterine cancer, hematopoietic tumor, or skin cancer.

### Item 4.

The antitumor agent according to any one of Items 1 to 3, wherein the malignant tumor patient expressing EGFR having exon 20 insertion mutation is a lung cancer patient.

### Item 5.

The antitumor agent according to any one of Items 1 to 4, wherein the exon 20 insertion mutation is a mutation in which one or more amino acids are inserted in the exon 20 region.

### Item 6.

The antitumor agent according to any one of Items 1 to 5, wherein the exon 20 insertion mutation is a mutation in which 1 to 7 amino acids are inserted in the exon 20 region.

### Item 7.

The antitumor agent according to any one of Items 1 to 6, wherein the exon 20 insertion mutation is a mutation in which 1 to 4 amino acids are inserted in the exon 20 region.

### Item 8.

The antitumor agent according to any one of Items 1 to 7, wherein the exon 20 insertion mutation is A763_Y764insFQEA, V769_D770insASV, D770_N771insSVD, D770_N771insNPG, D770_N771insG, D770>GY, N771_P772insN, P772_R773insPR, H773_V774insNPH, H773_V774insPH, H773_V774insAH, H773_V774insH, V774_C774insHV, or A761_E762insEAFQ.

### Item 9.

The antitumor agent according to any one of Items 1 to 8, wherein the exon 20 insertion mutation is V769_D770insASV, D770_N771insSVD, D770_N771insG, H773_V774insNPH, or H773_V774insPH.

### Item 10.

A method for treating a malignant tumor patient, comprising the step of administering an antitumor agent comprising an effective amount of a compound selected from the group consisting of.
(S)-N-(4-amino-6-methyl-5-(quinolin-3-yl)-8,9-dihydropyrimido[5,4-b]indolizin-8-yl)acrylamide;
(S)-N-(4-amino-6-methylene-5-(quinolin-3-yl)-7,8-dihydro-6H-pyrimido[5,4-b]pyrrolizin-7-yl)acrylamide;
(S,E)-N-(4-amino-6-methylene-5-(quinolin-3-yl)-7,8-dihydro-6H-pyrimido[5,4-b]pyrrolizin-7-yl)-3-chloroacrylamide; and
(R)-N-(4-amino-6-methyl-5-(quinolin-3-yl)-8,9-dihydropyrimido[5,4-b]indolizin-8-yl)-N-methylacrylamide, or a salt thereof, to a malignant tumor patient expressing EGFR having exon 20 insertion mutation.

### Item 11.

A compound selected from the group consisting of:
(S)-N-(4-amino-6-methyl-5-(quinolin-3-yl)-8,9-dihydropyrimido[5,4-b]indolizin-8-yl)acrylamide;
(S)-N-(4-amino-6-methylene-5-(quinolin-3-yl)-7,8-dihydro-6H-pyrimido[5,4-b]pyrrolizin-7-yl)acrylamide;
(S,E)-N-(4-amino-6-methylene-5-(quinolin-3-yl)-7,8-dihydro-6H-pyrimido[5,4-b]pyrrolizin-7-yl)-3-chloroacrylamide; and
(R)-N-(4-amino-6-methyl-5-(quinolin-3-yl)-8,9-dihydropyrimido[5,4-b]indolizin-8-yl)-N-methylacrylamide,
or a salt thereof, to treat a malignant tumor patient expressing EGFR having exon 20 insertion mutation.

### Item 12.

Use of a compound selected from the group consisting of:
(S)-N-(4-amino-6-methyl-5-(quinolin-3-yl)-8,9-dihydropyrimido[5,4-b]indolizin-8-yl)acrylamide;
(S)-N-(4-amino-6-methylene-5-(quinolin-3-yl)-7,8-dihydro-6H-pyrimido[5,4-b]pyrrolizin-7-yl)acrylamide; and
(S,E)-N-(4-amino-6-methylene-5-(quinolin-3-yl)-7,8-dihydro-6H-pyrimido[5,4-b]pyrrolizin-7-yl)-3-chloroacrylamide; and
(R)-N-(4-amino-6-methyl-5-(quinolin-3-yl)-8,9-dihydropyrimido[5,4-b]indolizin-8-yl)-N-methylacrylamide,
or a salt thereof for the production of an antitumor agent for treating a malignant tumor patient expressing EGFR having exon 20 insertion mutation.

### Advantageous Effects of Invention

The antitumor agent of the present invention exerts high selectivity with respect to exon 20 insertion mutant EGFR without inhibiting wild-type EGFR. Therefore, the antitumor agent of the present invention is useful in view of providing an antitumor agent that has reduced side effects derived from the inhibition of wild-type EGFR; and that exerts superior therapeutic effects for a malignant tumor patient expressing EGFR having exon 20 insertion mutation, for which the therapeutic effects of the previously known EGFR inhibitors are insufficient.

The previously known EGFR inhibitors have low selectivity with respect to exon 20 insertion mutant EGFR, compared with wild-type EGFR; therefore, the difference between the dosage for ensuring the antitumor effects and the dosage causing the side effects (skin disorders, digestive tract disorders, etc.) derived from wild-type EGFR inhibition was small. Accordingly, the previously known EGFR inhibitors have difficulty in exerting sufficient therapeutic effects. In contrast, since the antitumor agent of the present invention has high selectivity with respect to exon 20 insertion mutant EGFR, it is possible to increase the dosage without causing side effects derived from wild-type EGFR. Therefore, the antitumor agent of the present invention exerts superior therapeutic effects for a malignant tumor patient expressing EGFR having exon 20 insertion mutation.

### BRIEF DESCRIPTION OF DRAWINGS

FIG. 1 illustrates IC₅₀ ratios of wild-type EGFR to EGFR exon 20 insertion mutations calculated from the results of a test for cell growth inhibition on wild-type EGFR-expressing cell lines and mutant EGFR-expressing cell lines by compounds A, B, C, and D, a comparative compound, gefitinib, erlotinib, and afatinib.

FIG. 2 illustrates GI₅₀ ratios of wild-type EGFR to EGFR exon 20 insertion mutations calculated from the results of a test for cell growth inhibition on wild-type EGFR-expressing human cell lines and mutant EGFR-expressing human cell lines by compound A, a comparative compound, gefitinib, erlotinib, and afatinib.

FIG. 3 illustrates the relative tumor volume (which hereinafter also referred to as “RTV”) of mouse models that were subcutaneously transplanted with mutant EGFR-expressing cell lines (NIH3T3-EGFRinsASV cells) to measure the antitumor effect of compound A.

FIG. 4 illustrates the body weight change after grouping of mouse models that were subcutaneously transplanted with mutant EGFR-expressing cell lines (NIH3T3-EGFRinsASV cells) to measure the toxicity of compound A.

FIG. 5 illustrates the relative tumor volume of mouse models that were subcutaneously transplanted with mutant EGFR-expressing cell lines (NIH3T3-EGFRinsSVD cells) to measure the antitumor effect of compound A.

FIG. 6 illustrates the body weight after grouping of mouse models that were subcutaneously transplanted with mutant EGFR-expressing cell lines (NIH3T3-EGFRinsSVD cells) to measure the toxicity of compound A.

FIG. 7 illustrates the relative tumor volume of mouse models that were subcutaneously transplanted with mutant EGFR-expressing cell lines (H1975-EGFRinsSVD cells) to measure the antitumor effect of compound A.

FIG. 8 illustrates the body weight after grouping of mouse models that were subcutaneously transplanted with mutant EGFR-expressing cell lines (H1975-EGFRinsSVD cells) to measure the toxicity of compound A.

FIG. 9 illustrates the tumor volume of mouse models that were subcutaneously transplanted with mutant EGFR-expressing cell lines (NIH3T3-EGFRinsNPH) to measure the antitumor effect of compound A.

FIG. 10 illustrates the body weight after grouping of mouse models that were subcutaneously transplanted with mutant EGFR-expressing cell lines (NIH3T3-EGFRinsNPH) to measure the toxicity of compound A.

FIG. 11 illustrates the tumor volume of rat models that were subcutaneously transplanted with mutant EGFR-expressing cell lines (H1975-EGFRinsSVD cells) to measure the antitumor effect of compound A.

FIG. 12 illustrates the body weight after grouping of rat models that were subcutaneously transplanted with mutant EGFR-expressing cell lines (H1975-EGFRinsSVD cells) to measure the toxicity of compound A.

FIG. 13 illustrates the tumor volume of mouse models that were subcutaneously transplanted with a tumor derived from a lung cancer patient who was positive for EGFR with mutation V769_D770insASV to measure the antitumor effect of compound A.

FIG. 14 illustrates the body weight after grouping of mouse models that were subcutaneously transplanted with a tumor derived from a lung cancer patient who was positive for EGFR with mutation V769_D770insASV to measure the toxicity of compound A of the present invention.

FIG. 15 illustrates the amino acid sequence of wild-type EGFR (SEQ ID NO: 1).

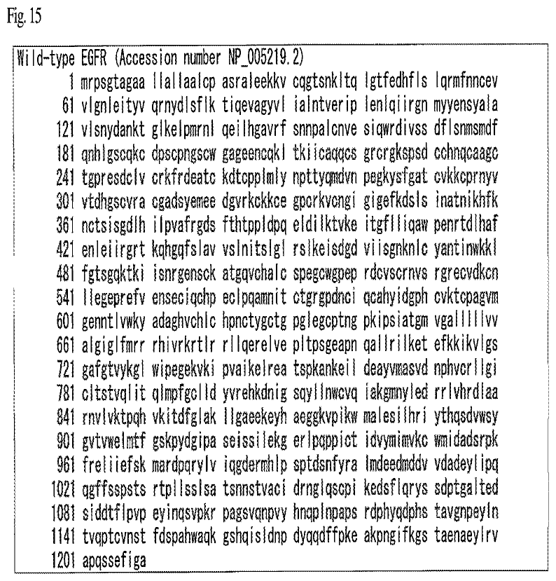

### Description of Embodiments

Preferable examples of various definitions in the scope of the present invention used in this specification are explained below in detail.

In this specification, “EGFR” refers to a human epidermal growth factor receptor protein, and is also referred to as ErbB-1 or HER1.

In this specification, “wild-type EGFR” refers to EGFR free of somatic mutation, which is a protein comprising the amino acid sequence represented by SEQ ID NO: 1 (GenBank accession number: NP_005219.2).

In this specification, “exon 20 insertion mutation” refers to a mutation in which one or more amino acids (preferably 1 to 7, more preferably 1 to 4) are inserted in the exon 20 region (the 761st to 823rd amino acid sequence in SEQ ID NO: 1) of EGFR, and is preferably a mutation in which amino acid sequence FQEA (phenylalanine, glutamine, glutamic acid, and alanine in this order from the N-terminus) is inserted between the 763rd alanine and 764th tyrosine in the exon 20 region (A763_Y764insFQEA); a mutation in which amino acid sequence ASV (alanine, serine, and valine in this order from the N-terminus) is inserted between the 769th valine and 770th aspartic acid in the exon 20 region (V769_D770insASV); a mutation in which amino acid sequence SVD (serine, valine, and aspartic acid in this order from the N-terminus) is inserted between the 770th aspartic acid and 771st asparagine in the exon 20 region (D770_N771insSVD); a mutation in which amino acid sequence NPG (asparagine, proline, and glycine in this order from the N-terminus) is inserted between the 770th aspartic acid and 771st asparagine in the exon 20 region (D770_N771insNPG); a mutation in which amino acid G (glycine) is inserted between the 770th aspartic acid and 771st asparagine (D770_N771insG); a mutation in which the 770th aspartic acid in the exon 20 region is deleted, and amino acid sequence GY (glycine and tyrosine in this order from the N-terminus) is inserted instead (D770>GY); a mutation in which amino acid N (asparagine) is inserted between the 771st asparagine and 772nd proline in the exon 20 region (N771_P772insN); a mutation in which amino acid sequence PR (proline and arginine in this order from the N-terminus) is inserted between the 772nd proline and 773rd histidine in the exon 20 region (P772_R773insPR); a mutation in which amino acid sequence NPH (asparagine, proline, and histidine in this order from the N-terminus) is inserted between the 773rd histidine and 774th valine in the exon 20 region (H773_V774insNPH); a mutation in which amino acid sequence PH (proline and histidine in this order from the N-terminus) is inserted between the 773rd histidine and 774th valine in the exon 20 region (H773_V774insPH); a mutation in which amino acid sequence AH (alanine and histidine in this order from the N-terminus) is inserted between the 773rd histidine and 774th valine in the exon 20 region (H773_V774insAH); a mutation in which amino acid H (histidine) is inserted between the 773rd histidine and 774th valine in the exon 20 region (H773_V774insH); a mutation in which amino acid sequence HV (histidine and valine in this order from the N-terminus) is inserted between the 774th valine and 775th cysteine in the exon 20 region (V774_C775insHV); a mutation in which amino acid sequence EAFQ (glutamic acid, alanine, phenylalanine, and glutamine in this order from the N-terminus) is inserted between the 761st alanine and 762nd glutamic acid in the exon 20 region (A761_E762insEAFQ); and the like. More preferable mutations include a mutation in which amino acid sequence ASV (alanine, serine, and valine in this order from the N-terminus) is inserted between the 769th valine and 770th aspartic acid in the exon 20 region (V769_D770insASV); a mutation in which amino acid sequence SVD (serine, valine, and aspartic acid in this order from the N-terminus) is inserted between the 770th aspartic acid and 771st asparagine in the exon 20 region (D770_N771insSVD); a mutation in which amino acid G (glycine) is inserted between the 770th aspartic acid and 771th asparagine in the exon 20 region (D770_N771insG); a mutation in which amino acid sequence NPH (asparagine, proline, and histidine in this order from the N-terminus) is inserted between the 773rd histidine and 774th valine in the exon 20 region (H773_V774insNPH); and a mutation in which amino acid sequence PH (proline and histidine in this order from the N-terminus) is inserted between the 773rd histidine and 774th valine in the exon 20 region (H773_V774insPH). More preferable mutations include a mutation in which amino acid sequence SVD (serine, valine, and aspartic acid in this order from the N-terminus) is inserted between the 770th aspartic acid and 771st asparagine in the exon 20 region (D770_N771insSVD); and a mutation in which amino acid G (glycine) is inserted between the 770th aspartic acid and 771st asparagine in the exon 20 region (D770_N771insG).

In this specification, the “malignant tumor patient expressing EGFR having exon 20 insertion mutation” refers to a malignant tumor patient expressing EGFR having exon 20 insertion mutation in at least one part of the exon 20 region of EGFR. The EGFR may have exon 20 insertion mutation in two or more different parts, but preferably one part thereof. Further, the EGFR may also have a mutation other than exon 20 insertion mutation (such as exon 19 deletion mutation, L858R mutation, or L790M mutation).

In the present invention, the method for detecting exon 20 insertion mutation of EGFR expressed in a malignant tumor patient is not particularly limited insofar as the method is capable of detecting the mutation, and any known detection methods may be used. The detection target in the detection of exon insertion mutation may be any of genome sequence of EGFR gene, transcriptional product of EGFR gene, and EGFR protein.

The sample used in the detection of exon 20 insertion mutation is not particularly limited as long as the sample is a biological sample isolated from a malignant tumor patient, in particular, a sample that is obtained from a malignant tumor patient and contains malignant tumor cells. Examples of biological samples include body fluids (e.g., blood, urine, etc.), tissues, the extracts thereof, and the cultures of obtained tissues. The method for isolating a biological sample can be suitably selected depending on the type of biological sample.

The biological sample is prepared by being appropriately treated according to the detection method. Further, the reagent used for the detection (e.g., a reagent containing primer or probe) may be prepared by a conventional method according to the detection method.

In one embodiment of the present invention, the step for detecting the presence of exon 20 insertion mutation of EGFR expressed in a malignant tumor patient may be performed before the administration of antitumor agent to a malignant tumor patient.

Compounds A to D (Compounds A, B, C, and D) (in this specification, these compounds may also be generally referred to as a “compound of the present invention” or a “compound according to the present invention”) and the production method thereof are explained below.

Compound A ((S)-N-(4-amino-6-methyl-5-(quinolin-3-yl)-8,9-dihydropyrimido[5,4-b]indolizin-8-yl)acrylamide) is represented by the following chemical formula.

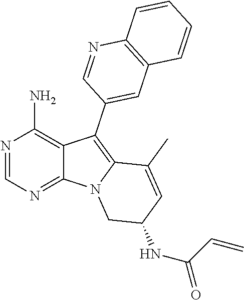 [SMILES: C=CC(=O)N[C@H]1C=C(C)c2c(-c3cnc4ccccc4c3)c3c(N)ncnc3n2C1]

Compound B ((S)-N-(4-amino-6-methylene-5-(quinolin-3-yl)-7,8-dihydro-6H-pyrimido[5,4-b]pyrrolizin-7-yl)acrylamide) is represented by the following chemical formula.

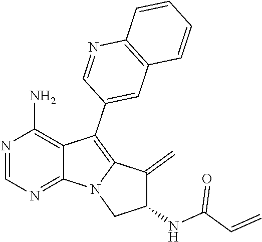 [SMILES: C=CC(=O)N[C@@H]1Cn2c(c(-c3cnc4ccccc4c3)c3c(N)ncnc32)C1=C]

Compound C ((S,E)-N-(4-amino-6-methylene-5-(quinolin-3-yl)-7,8-dihydro-6H-pyrimido[5,4-b]pyrrolizin-7-yl)-3-chloroacrylamide) is represented by the following chemical formula.

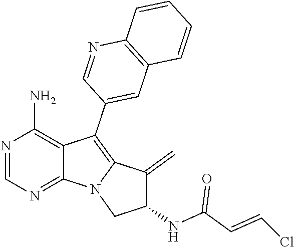 [SMILES: C=C1c2c(-c3cnc4ccccc4c3)c3c(N)ncnc3n2C[C@H]1NC(=O)/C=C/Cl]

Compound D ((R)-N-(4-amino-6-methyl-5-(quinolin-3-yl)-8,9-dihydropyrimido[5,4-b]indolizin-8-yl)-N-methylacrylamide) is represented by the following chemical formula.

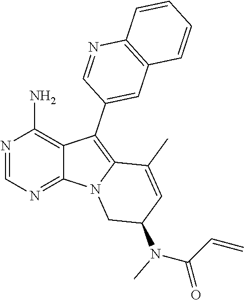 [SMILES: C=CC(=O)N(C)[C@@H]1C=C(C)c2c(-c3cnc4ccccc4c3)c3c(N)ncnc3n2C1]

Compounds A to D may be produced, for example, through the production method disclosed in WO2015/025936A1, the methods described in the Examples, and the like. However, the production methods of Compounds A to D are not limited to these reaction examples.

When Compounds A to D of the present invention have isomers such as optical isomers, stereoisomers, rotational isomers, and tautomers, any of the isomers and mixtures thereof are included within the scope of the compound of the present invention, unless otherwise specified. For example, when Compounds A to D of the present invention have optical isomers, racemic mixtures and the optical isomers separated from a racemic mixture are also included within the scope of the compound of the present invention, unless otherwise specified.

The salts of Compounds A to D refer to any pharmaceutically acceptable salts; examples include base addition salts and acid addition salts.

Examples of base addition salts include alkali metal salts such as sodium salts and potassium salts; alkaline earth metal salts such as calcium salts and magnesium salts; ammonium salts; and organic amine salts such as trimethylamine salts, triethylamine salts, dicyclohexylamine salts, ethanolamine salts, diethanolamine salts, triethanolamine salts, procaine salts, and N,N′-dibenzylethylenediamine salts.

Examples of acid addition salts include inorganic acid salts such as hydrochlorides, sulfates, nitrates, phosphates, and perchlorates; organic acid salts such as acetates, formates, maleates, fumarates, tartrates, citrates, ascorbates, and trifluoroacetates; and sulfonates such as methanesulfonates, isethionates, benzenesulfonates, and p-toluenesulfonates.

Compounds A to D and salts thereof also encompass prodrugs thereof. A prodrug refers to a compound that can be converted to Compounds A to D or a salt thereof through a reaction with an enzyme, gastric acid, or the like, under physiological conditions in vivo, i.e., a compound that can be converted to the compound of the present invention or a salt thereof by enzymatic oxidation, reduction, hydrolysis, or the like; or a compound that can be converted to Compounds A to D or a salt thereof by hydrolysis or the like with gastric acid or the like. Further, the prodrug may be compounds that can be converted to Compounds A to D or a salt thereof under physiological conditions, such as those described in “Iyakuhin no Kaihatsu [Development of Pharmaceuticals],” Vol. 7, Molecular Design, published in 1990 by Hirokawa Shoten Co., pp. 163-198.

### Description of Diseases

Specific examples of tumors targeted in the present invention include, but are not particularly limited to, head and neck cancer, gastrointestinal cancer (esophageal cancer, stomach cancer, duodenal cancer, liver cancer, biliary cancer (e.g., gallbladder and bile duct cancer), pancreatic cancer, colorectal cancer (e.g., colon cancer, and rectal cancer), etc.), lung cancer (e.g., non-small-cell lung cancer, small-cell lung cancer, and mesothelioma), breast cancer, genital cancer (ovarian cancer, uterine cancer (e.g., cervical cancer, and endometrial cancer), etc.), urological cancer (e.g., kidney cancer, bladder cancer, prostate cancer, and testicular tumor), hematopoietic tumor (e.g., leukemia, malignant lymphoma, and multiple myeloma), osteosarcoma, soft-tissue sarcoma, skin cancer, brain tumor, and the like. Preferable examples include lung cancer, breast cancer, head and neck cancer, brain tumor, uterine cancer, hematopoietic tumor, or skin cancer.

When Compounds A to D or a salt thereof are used as a pharmaceutical agent, a pharmaceutical carrier can be added, if required, thereby forming a suitable dosage form according to prevention and treatment purposes. Examples of the dosage form include oral preparations, injections, suppositories, ointments, patches, and the like. Oral preparations are preferable. Such dosage forms can be formed by methods conventionally known to persons skilled in the art.

As the pharmaceutical carrier, various conventional organic or inorganic carrier materials used as preparation materials may be blended as an excipient, binder, disintegrant, lubricant, or colorant in solid preparations; or as a solvent, solubilizing agent, suspending agent, isotonizing agent, buffer, or soothing agent in liquid preparations. Moreover, pharmaceutical preparation additives, such as antiseptics, antioxidants, colorants, sweeteners, and stabilizers, may also be used, if required.

Oral solid preparations are prepared as follows. After an excipient is added optionally with a binder, disintegrant, lubricant, colorant, taste-masking or flavoring agent, etc., to Compounds A to D, the resulting mixture is formulated into tablets, coated tablets, granules, powders, capsules, or the like by ordinary methods.

Examples of excipients include lactose, sucrose, D-mannitol, glucose, starch, calcium carbonate, kaolin, microcrystalline cellulose, and silicic acid anhydride. Examples of binders include water, ethanol, 1-propanol, 2-propanol, simple syrup, liquid glucose, liquid a-starch, liquid gelatin, D-mannitol, carboxymethyl cellulose, hydroxypropyl cellulose, hydroxypropyl starch, methyl cellulose, ethyl cellulose, shellac, calcium phosphate, polyvinylpyrrolidone, and the like. Examples of disintegrators include dry starch, sodium alginate, powdered agar, sodium hydrogen carbonate, calcium carbonate, sodium lauryl sulfate, stearic acid monoglyceride, lactose, and the like. Examples of lubricants include purified talc, sodium stearate, magnesium stearate, borax, polyethylene glycol, and the like. Examples of colorants include titanium oxide, iron oxide, and the like. Examples of taste-masking or flavoring agents include sucrose, bitter orange peel, citric acid, tartaric acid, and the like.

When a liquid preparation for oral administration is prepared, a taste-masking agent, a buffer, a stabilizer, a flavoring agent, and the like may be added to Compounds A to D; and the resulting mixture may be formulated into an oral liquid preparation, syrup, elixir, etc., according to an ordinary method.

Examples of taste-masking or flavoring agents include those mentioned above. Examples of buffer agents include sodium citrate and the like. Examples of stabilizers include tragacanth, gum arabic, gelatin, and the like. As necessary, these preparations for oral administration may be coated according to methods known in the art with an enteric coating or other coating for the purpose of, for example, persistence of effects. Examples of such coating agents include hydroxypropyl methylcellulose, ethyl cellulose, hydroxymethyl cellulose, hydroxypropyl cellulose, polyoxyethylene glycol, and Tween 80 (registered trademark).

When an injection agent is prepared, a pH regulator, a buffer, a stabilizer, an isotonizing agent, a local anesthetic, and the like, may be added to Compounds A to D; and the mixture may be formulated into a subcutaneous, intramuscular, or intravenous injection according to an ordinary method.

Examples of the pH adjuster and the buffer used herein include sodium citrate, sodium acetate, and sodium phosphate. Examples of the stabilizer include sodium pyrosulfite, EDTA, thioglycolic acid, and thiolactic acid. Examples of the local anesthetic include procaine hydrochloride and lidocaine hydrochloride. Examples of the tonicity agent include sodium chloride, glucose, D-mannitol, and glycerol.

When a suppository is prepared, pharmaceutically acceptable carriers known by a person skilled in the art, such as polyethylene glycol, lanolin, cacao butter, and fatty acid triglyceride; and as necessary, surfactants such as Tween 80 (registered trademark), may be added to Compounds A to D, and the resulting mixture may be formulated into a suppository according to an ordinary method.

When an ointment is prepared, a commonly used base, stabilizer, wetting agent, preservative, and the like, may be blended into Compounds A to D, as necessary; and the obtained mixture may be mixed and formulated into an ointment according to an ordinary method.

Examples of the base include liquid paraffin, white petrolatum, white beeswax, octyl dodecyl alcohol, and paraffin.

Examples of the preservative include methyl paraoxybenzoate, ethyl paraoxybenzoate, and propyl paraoxybenzoate.

When a patch is prepared, the above-described ointment, cream, gel, paste, or the like, may be applied to an ordinary substrate according to an ordinary method.

Examples of substrates include woven fabrics or non-woven fabrics comprising cotton, staple fibers, or chemical fibers; and films or foam sheets of soft vinyl chloride, polyethylene, polyurethane, etc., are suitable.

The amount of Compounds A to D to be incorporated in each of such dosage unit forms depends on the condition of the patient to whom the compound is administered, the dosage form thereof, etc. In general, in the case of an oral agent, the amount of the compound is preferably 0.05 to 1000 mg per dosage unit form. In the case of an injection, the amount of the compound is preferably 0.01 to 500 mg per dosage unit form; and in the case of a suppository, the amount of the compound is preferably 1 to 1000 mg per dosage unit form.

Further, the daily dose of the medicine in such a dosage form depends on the condition, body weight, age, sex, etc., of the patient, and cannot be generalized. For example, the daily dose for an adult (body weight: 50 kg) of Compounds A to D as an active ingredient may be generally 0.05 to 5000 mg, and preferably 0.1 to 1000 mg; and is preferably administered in one dose, or in two to three divided doses, per day.

The present invention also provides a method for treating a malignant tumor patient, comprising the step of administering an effective amount of an antitumor agent comprising a compound selected from the group consisting of Compounds A to D or a salt thereof to a malignant tumor patient expressing EGFR having exon 20 insertion mutation.

The present invention also provides a compound selected from the group consisting of Compounds A to D, or a salt thereof for treating a malignant tumor patient expressing EGFR having exon 20 insertion mutation.

The present invention also provides use of a compound selected from the group consisting of Compounds A to D or a salt thereof for treating a malignant tumor patient expressing EGFR having exon 20 insertion mutation.

The present invention also provides use of a compound selected from the group consisting of Compounds A to D or a salt thereof for the production of an antitumor agent for treating a malignant tumor patient expressing EGFR having exon 20 insertion mutation.

The present invention is also a method for predicting therapeutic effects of chemotherapy using an antitumor agent comprising, as an active ingredient, a compound selected from the group consisting of Compounds A to D or a salt thereof in a malignant tumor patient, the method comprising steps (1) and (2) below:
(1) a step of detecting the presence or absence of mutation of EGFR gene contained in a biological sample obtained from the patient; and
(2) a step of predicting that the chemotherapy is highly likely to exhibit sufficient therapeutic effects with respect to the patient when the results of the detection in step (1) found that the EGFR gene has exon 20 insertion mutation.

The present invention is also a method for treating a malignant tumor patient, the method comprising steps (1) to (3) below:
(1) a step of detecting the presence or absence of mutation of EGFR gene contained in a biological sample obtained from the patient;
(2) a step of predicting that the chemotherapy using an antitumor agent comprising a compound selected from the group consisting of Compounds A to D or a salt thereof is highly likely to exhibit sufficient therapeutic effects with respect to the patient when the results of the detection in step (1) found that the EGFR gene has exon 20 insertion mutation; and
(3) a step of administering the antitumor agent to a patient who was predicted highly likely to sufficiently respond to the chemotherapy in step (2).

The base sequence of EGFR gene is publicly known. The GenBank accession number of the base sequence of cDNA is NM_005228.4.

The “therapeutic effects” can be evaluated by tumor shrinkage effects, relapse-suppressing effects, life-prolonging effects, and the like. The relapse-suppressing effects may be shown as degree of the extension of non-relapse period and/or the degree of the improvement in relapse rate; and the life-prolonging effects may be shown as the degree of the entire survival time and/or the degree of the extension of the median of progression-free survival, or the like. The “sufficient therapeutic effects” of the chemotherapy using an antitumor agent comprising, as an active ingredient, Compound A or a salt thereof means that superior therapeutic effects are obtained by the administration of the antitumor agent comprising, as an active ingredient, Compound A or a salt thereof, such as significant extension of survival time, significant suppression of relapse, and the like, compared with non-administration.

### EXAMPLES

The following describes the present invention in more detail with reference to the following Test Examples. However, the present invention is not limited to these Examples (Test Examples).

### Test Example 1

### In Vitro Drug Efficacy Test

### Evaluation of Cell Growth Inhibitory Effect on Wild-Type EGFR- or Mutant EGFR-Expressing Cell Lines (1)

The inhibitory activity of compounds against wild-type EGFR and mutant EGFR was evaluated using Ba/F3 cells (mouse B-lymphocyte precursor cell lines) to which human EGFR genes were introduced. The Ba/F3 cells were maintained in an RPMI-1640 medium (Thermo Fisher Scientific) containing 10% fetal bovine serum (FBS), 100 U/mL penicillin/100 μg/mL streptomycin (Thermo Fisher Scientific), and 1 ng/mL mouse interleukin-3 (mlL-3) (CST). A PB-CMV-MCS-EF1-GFP+Puro vector or PB-CMV-MCS-EF1-RFP+Puro vector into which a human EGFR gene (wild-type (WT), V769_D770insASV (insASV), D770_N771insSVD (insSVD), D770_N771insG (insG), H773_V774insNPH (insNPH), or H773_V774insPH (insPH)) was encoded was introduced to the cells, together with a Super PiggyBac transposase expression vector, by electroporation using an Amaxa (trademark) Cell Line Nucleofector (trademark) Kit V, followed by selection using puromycin (SIGMA). Ba/F3 cells expressing wild-type EGFR (which hereinafter also referred to as “Ba/F3-EGFR_WT”) exhibited mlL-3-independent growth in the presence of 50 ng/mL EGF (R&D Systems); and Ba/F3 cells expressing EGFR exon 20 insertion mutation (which hereinafter also referred to as “Ba/F3-EGFRinsASV,” “Ba/F3-EGFRinsSVD,” “Ba/F3-EGFRinsG,” “Ba/F3-EGFRinsNPH,” or “Ba/F3-EGFRinsPH”) exhibited mlL-3-independent growth in the absence of EGF.

To evaluate the cell growth inhibitory effect, Ba/F3-EGFR_WT cells were suspended in an RPMI-1640 medium containing 10% FBS, 100 U/mL penicillin, 100 μg/mL streptomycin, and 50 ng/mL EGF; and the cell suspension was seeded in each well of a 96-well flat-bottom microplate such that the cell count per well was 30,000. The Ba/F3 cells expressing EGFR exon 20 insertion mutation were suspended in an RPMI-1640 medium containing 10% FBS, 100 U/mL penicillin, and 100 μg/mL streptomycin; and the cell suspension was seeded in each well of a 96-well flat-bottom microplate such that the cell count per well was 15,000. Subsequently, (S)-N-(4-amino-6-methyl-5-(quinolin-3-yl)-8,9-dihydropyrimido[5,4-b]indolizin-8-yl)acrylamide (compound A), (S)-N-(4-amino-6-methylene-5-(quinolin-3-yl)-7,8-dihydro-6H-pyrimido[5,4-b]pyrrolizin-7-yl)acrylamide (compound B), (S,E)-N-(4-amino-6-methylene-5-(quinolin-3-yl)-7,8-dihydro-6H-pyrimido[5,4-b]pyrrolizin-7-yl)-3-chloroacrylamide (compound C), and (R)-N-(4-amino-6-methyl-5-(quinolin-3-yl)-8,9-dihydropyrimido[5,4-b]indolizin-8-yl)-N-methylacrylamide (compound D) prepared in accordance with the production method disclosed in PTL 2, and (S)-N-(4-amino-5-(quinolin-3-yl)-6,7,8,9-tetrahydropyrimido[5,4-b]indolizin-8-yl)acrylamide prepared in accordance with the production method disclosed in WO2013/125709A1 (the compound of Example 1 in WO2013/125709A1, which hereinafter also referred to as “comparative compound”) were dissolved in DMSO, and diluted with DMSO or the medium used for suspending the cells. These compounds were individually added to each well of the culture plate of the cells, and incubated in a 5% CO₂ gas-containing incubator at 37° C. for 3 days. The cell count after incubation was measured using a CellTiter-Glo (trademark) Luminescent Cell Viability Assay (Promega Corporation) in accordance with the manufacturer's recommended protocol. The growth rate was calculated using the following formula, and the concentration of each test compound for 50% inhibition (IC₅₀(μM)) was determined.

Growth Rate (%)=T/C×100
T: the luminescence intensity of a well to which a test compound was added.
C: the luminescence intensity of a well to which the test compound was not added.

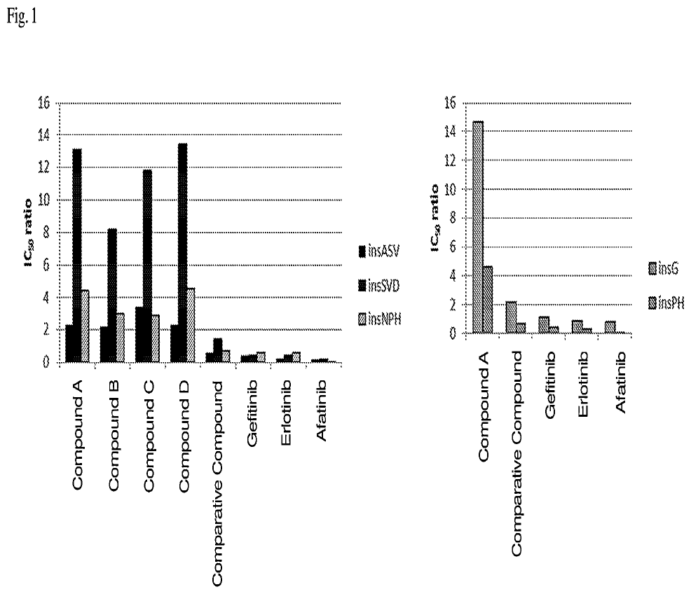

Additionally, the ratio of IC₅₀ between wild-type EGFR and EGFR exon 20 insertion mutation was determined using the following formula. FIG. 1 illustrates the results.

IC₅₀Ratio=IC₅₀(WT)/IC₅₀(ex20ins)
IC₅₀(WT): IC₅₀ for wild-type EGFR
IC₅₀(ex20ins): IC₅₀ for EGFR exon 20 insertion mutation

As is clear from FIG. 1, compounds A to D exhibited a cell growth inhibitory effect on cell lines expressing EGFR exon 20 insertion mutations; and their mutation selectivity was higher than that of the comparative compound, gefitinib, erlotinib, and afatinib.

### Test Example 2

### Cell Growth Inhibitory Effect on Wild-Type EGFR- or Mutant EGFR-Expressing Human Cell Lines (2)

To evaluate the inhibitory activity of compounds against wild-type EGFR and mutant EGFR, the following cells were used: NCI-H1975 cells, which are human pulmonary adenocarcinoma cell lines expressing EGFR with mutation D770_N771insSVD by gene modification (which hereinafter also referred to as “H1975-EGFRinsSVD”); and A431 cells, which are human epithelial cancer cell lines expressing wild-type EGFR. H1975-EGFRinsSVD cells were prepared as follows. A PB-CMV-MCS-EF1-RFP+Puro vector into which D770_N771insSVD (insSVD) was encoded was introduced into NCI-H1975 cells, together with a Super PiggyBac Transposase expression vector, by electroporation using an Amaxa (trademark) Cell Line Nucleofector (trademark) Kit R, followed by selection using puromycin (SIGMA). XTN (trademark) TALENs Site-Specific Nucleases (Transposagen) were introduced into the cells by electroporation using the Amaxa (trademark) Cell Line Nucleofector (trademark) Kit R, and endogenous-EGFR (T790M/L858R)-knockout cells were selected by sequencing.

To evaluate the cell growth inhibitory effect, individual types of cells were suspended in a medium recommended by ATCC. The cell suspensions were seeded in each well of respective 96-well flat-bottom plates such that the cell count per well was 3,000, and incubated in a 5% CO₂-containing incubator at 37° C. for 1 day. Compound A, the comparative compound, gefitinib, erlotinib, and afatinib were individually dissolved in DMSO, and diluted with DMSO such that these test compounds have a concentration 200 times higher than the final concentration. These DMSO solutions of the test compounds were diluted with the medium used for suspending the cells, and added to each well of the culture plates of the cells such that DMSO has a final concentration of 0.5%, and the cells were incubated in a 5% CO₂-containing incubator at 37° C. for 3 days. The cell count at the time incubation started (day 0) and the cell count after incubation (day 3) were measured using a CellTiter-Glo (trademark) Luminescent Cell Viability Assay (Promega Corporation) in accordance with the manufacturer's recommended protocol. The growth rate was calculated using the following formula, and the concentration of each test compound for 50% inhibition (GI₅₀(μM)) was determined. Table 1 illustrates the results.

### 1) If T on day 3≥C on day 0:

Growth Rate (%)=(T on day 3−C on day 0)/(C on day 3−C on day 0)×100
T: the luminescence intensity of a well to which a test compound was added.
C: the luminescence intensity of a well to which the test compound was not added.
Day 0: the day on which a test compound was added.
Day 3: the day on which evaluation was performed.

### 2) If T on day 3<C on day 0:

Growth Rate (%)=(T on day 3−C on day 0)/(C on day 0)×100
T: the luminescence intensity of a well to which a test compound was added.
C: the luminescence intensity of a well to which the test compound was not added.
Day 0: the day on which a test compound was added.
Day 3: the day on which evaluation was performed.

TABLE 1
GI₅₀ (μM)

| | A431 | H1975 EGFRinsSVD |
|---|---|---|
| Compound A | 0.396 | 0.031 |
| Comparative Compound | 0.543 | 0.364 |
| Gefitinib | 0.310 | 1.903 |
| Erlotinib | 0.612 | 2.775 |
| Afatinib | 0.023 | 0.189 |
| Osimertinib | 0.321 | 0.194 |

Additionally, the ratio of GI₅₀ between wild-type EGFR and EGFR exon 20 insertion mutation was determined using the following formula. Table 2 illustrates the results.

GI₅₀Ratio=GI₅₀(A431)/GI₅₀(H1975 EGFRinsSVD)
GI₅₀(A431): GI₅₀ for wild-type EGFR
GI₅₀(H1975 EGFRinsSVD): GI₅₀ for EGFR exon 20 insertion mutation

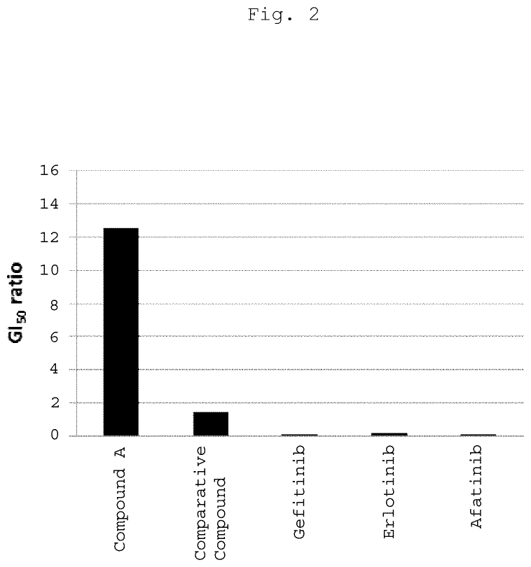

As is clear from Table 1 and FIG. 2, compound A exhibited an cell growth inhibitory effect on cell lines expressing EGFR exon 20 insertion mutation, and its mutation selectivity was higher than that of the comparative compound, gefitinib, erlotinib, afatinib, and osimertinib.

### Test Example 3

### Evaluation of Phosphorylated EGFR Inhibitory Activity Against Wild-Type EGFR- or Mutant EGFR-Expressing Cell Lines (1)

A431 cells, which are human epithelial cancer cell lines overexpressing wild-type EGFR and H1975-EGFRinsSVD cells, which are human pulmonary adenocarcinoma cell lines expressing EGFR with mutation D770_N771insSVD by gene modification, were suspended in respective mediums. These cell suspensions were individually seeded into a 60-mm dish, and incubated in a 5% CO₂-containing incubator at 37° C. for 1 day. Compound A was dissolved in DMSO, and diluted with DMSO such that the test compound has a concentration 1000 times higher than the final concentration. The DMSO solution of the test compound was diluted with each medium used for suspending the cells, and each diluted solution was added to respective culture dishes of the cells such that DMSO has a final concentration of 0.1%, followed by incubation in a 5% CO₂-containing incubator at 37° C. for 6 hours. After incubation, the cells were collected, and stored at −80° C. in the form of pellets until use. A RIPA buffer (Thermo Fisher Scientific) containing a protease inhibitor cocktail (Thermo Fisher Scientific) was added to the pellets, and proteins within the cells were extracted. The concentration of the proteins were measured using a BCA protein assay kit (Thermo Fisher Scientific), and each sample was adjusted so as to have a protein concentration suitable for measurement of phosphorylated EGFR expression. The phosphorylated EGFR expression was measured using a Simple Western (trademark) assay system (ProteinSimple) in accordance with the manufacturer's recommended protocol. The primary antibody used in measurement was a Phospho-EGF Receptor (Tyr1068) #3777 (CST) diluted to 1/50.

For each type of cells, a calibration curve of the protein concentration (x axis) and the phosphorylated EGFR expression level (y axis) was prepared, and the phosphorylated EGFR expression level of each sample was converted to a protein concentration based on the calibration curve. The phosphorylated EGFR rate was calculated using the following formula to determine the concentration of the test compound at which phosphorylated EGFR was inhibited by 50% (IC₅₀(μM)).

Phosphorylated EGFR Rate (%)=T/C×100
T: an equivalent amount for the protein concentration of a sample to which the test compound was added.
C: an equivalent amount for the protein concentration of a sample to which the test compound was not added.

Additionally, the selectivity for wild-type EGFR and EGFR exon 20 insertion mutation was calculated using the following formula. Table 2 illustrates the results.

IC₅₀Ratio=IC₅₀(A431)/IC₅₀(H1975 EGFRinsSVD)
IC₅₀(A431): IC₅₀ for wild-type EGFR
IC₅₀(H1975 EGFRinsSVD): IC₅₀ for EGFR exon 20 insertion mutation

TABLE 2

| | IC₅₀ (μM) | |
|---|---|---|
| | A431 | H1975 EGFRinsSVD |
| IC₅₀ (μM) | 0.535 | 0.023 |
| IC₅₀ Ratio | — | 23.3 |

As is clear from Table 2, compound A exhibited a selective inhibition activity against EGFR exon 20 insertion mutation.

### Test Example 4

### Evaluation of Phosphorylated EGFR Inhibitory Activity Against Wild-Type EGFR- or Mutant EGFR-Expressing Cell Lines (2)

The autophosphotylation inhibitory activity of a compound against wild-type EGFR and mutant EGFR was evaluated using NIH-3T3 cells, which are mouse fibroblast cell lines to which human EGFR gene was introduced. NIH-3T3 cells were maintained in a D-MEM (high-glucose) medium (Wako Pure Chemical Industries, Ltd.) containing 10% newborn calf serum (NBCS), 1,500 mg/L sodium hydrogen carbonate, and 100 U/mL penicillin/100 μg/mL streptomycin (Thermo Fisher Scientific). A PB-CMV-MCS-EF1-RFP+Puro vector into which a human EGFR gene (WT, insASV, insSVD, insG, insNPH, or insPH) was encoded was introduced into the cells, together with a Super PiggyBac Transposase expression vector, by electroporation using an Amaxa (trademark) Cell Line Nucleofector (trademark) Kit R, followed by selection using puromycin (SIGMA). NIH-3T3 cells expressing wild-type EGFR (which hereinafter also referred to as “NIH3T3-EGFR_WT”) exhibited growth in the presence of 50 ng/mL EGF (R&D Systems) under 1% NBCS conditions. NIH-3T3 cells expressing EGFR exon 20 insertion mutation (which hereinafter also referred to as “NIH3T3-EGFRinsASV,” “NIH3T3-EGFRinsSVD,” “NIH3T3-EGFRinsG,” “NIH3T3-EGFRinsNPH,” or “NIH3T3-EGFRinsPH”) exhibited growth in the absence of EGF under 1% NBCS conditions.

To evaluate EGFR-autophosphorylation inhibitory activity, NIH3T3 cells to which human EGFR was introduced were suspended in respective mediums. These cell suspensions were individually seeded into a 60-mm dish or 6-well flat-bottom plate, and incubated in a 5% CO₂-containing incubator at 37° C. for 1 day. Compound A was dissolved in DMSO, and diluted with DMSO such that the test compound has a concentration 400 times higher than the final concentration. The DMSO solutions of the test compound were diluted with the medium used for suspending the cells, and added to the culture dishes of the cells such that DMSO has a final concentration of 0.25%. Further, EGF was added to the culture dish of NIH3T3-EGFR_WT cells to give a final concentration of 50 ng/mL. The culture dishes were all subjected to incubation in a 5% CO₂-containing incubator at 37° C. for 6 hours. After incubation, the cells were collected, and stored at −80° C. in the form of pellets until use. A RIPA buffer (Thermo Fisher Scientific) containing a protease inhibitor cocktail (Thermo Fisher Scientific) was added to the pellets, and proteins within the cells were extracted. The protein concentration was measured using a BCA protein assay kit (Thermo Fisher Scientific), and each sample was adjusted to a protein concentration suitable for measurement of phosphorylated EGFR expression. The phosphorylated EGFR expression was measured using a Simple Western (trademark) assay system (ProteinSimple) in accordance with the manufacturer's recommended protocol. The primary antibody used in measurement was a Phospho-EGF Receptor (Tyr1068) #3777 (CST) diluted to 1/50.

For each type of cells, a calibration curve of the protein concentration (x axis) and the phosphorylated EGFR expression level (y axis) was prepared, and the phosphorylated EGFR expression level of each sample was converted to a protein concentration based on the calibration curve. The phosphorylated EGFR inhibitory rate was calculated using the following formula to determine the concentration of the test compound at which phosphorylated EGFR was inhibited by 50% (IC₅₀(μM)).

Phosphorylated EGFR Inhibitory Rate (%)=T/C×100
T: an equivalent amount for the protein concentration of a sample to which the test compound was added.
C: an equivalent amount for the protein concentration of a sample to which the test compound was not added.

Additionally, the selectivity for wild-type EGFR and EGFR exon 20 insertion mutation was calculated using the following formula. Table 3 illustrates the results.

IC₅₀Ratio=IC₅₀(WT)/IC₅₀(EGFR exon 20 insertion mutation)

TABLE 3

| | WT | insASV | insSVD | insG | insNPH | insPH |
|---|---|---|---|---|---|---|
| IC₅₀ (μM) | 0.571 | 0.197 | 0.033 | 0.073 | 0.083 | 0.159 |
| IC₅₀ Ratio | — | 2.9 | 17.1 | 7.9 | 6.9 | 3.6 |

As is clear from Table 3, compound A exhibited a selective inhibition activity against various EGFR exon 20 insertion mutations.

As is clear from the results of Test Examples 1 to 4, compounds A to D exhibited a cell growth inhibitory effect, accompanied by an EGFR inhibitory effect, on cell lines expressing EGFR exon 20 insertion mutation; and the effect and mutation selectivity were higher than those of the comparative compound, gefitinib, erlotinib, afatinib, and osimertinib.

### Test Example 5

### In Vivo Drug Efficacy Test

Evaluation of Antitumor Effect on Model Subcutaneously Transplanted with Mutant EGFR-Expressing Cell Lines

Nude mice were subcutaneously transplanted with NIH3T3-EGFRinsASV cells, NIH3T3-EGFRinsSVD cells, or H1975-EGFRinsSVD cells to which human mutant EGFR was introduced. At the point at which the tumor volume of the tumor engrafted in the nude mice grew to about 100 to 200 mm³, the mice were allocated into groups, 5 to 6 mice for each group, by stratified randomization such that the average tumor volume between the groups was uniform. The mice were then orally administered compound A or afatinib once daily for 14 consecutive days.

The dose of afatinib was 20 mg/kg/day, which is the maximum tolerated dose (the highest dose at which the weight loss during a dosing period is less than 20%) for 14 days, a dosing period of this test; and the dose of compound A was 200 mg/kg/day (maximum tolerated dose). The maximum tolerated dose was determined in accordance with the “Guidelines Involving Experimental Neoplasia Proposals in Mice and Rats” of the National Cancer Institute (NCI), from a humanitarian perspective.

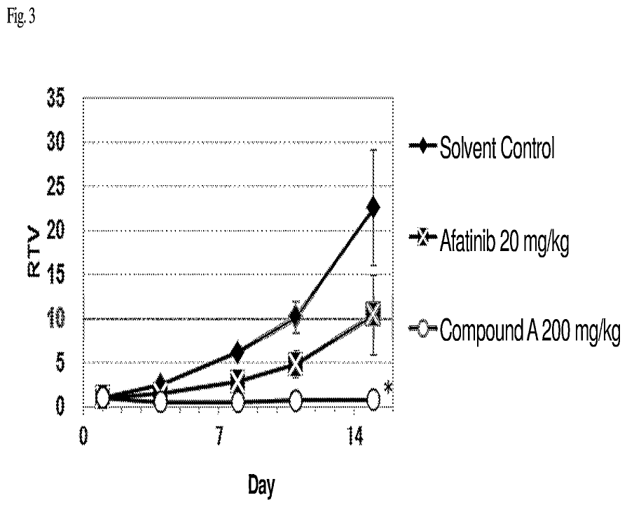

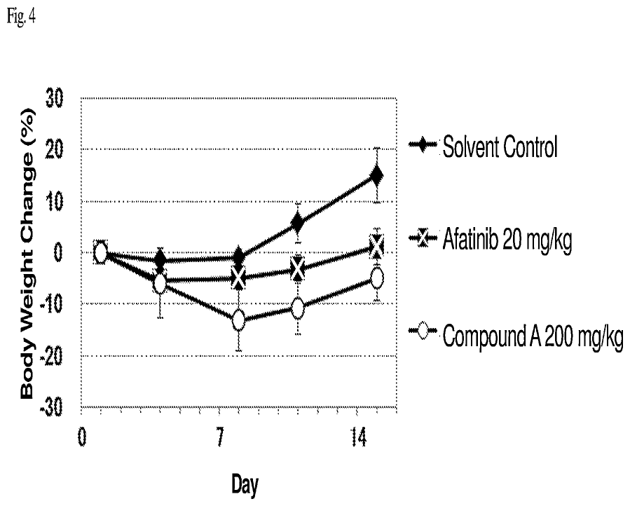

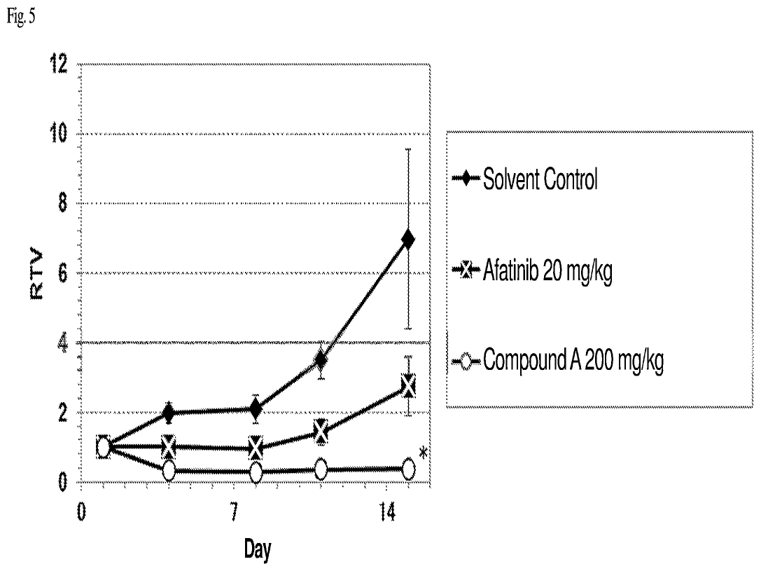

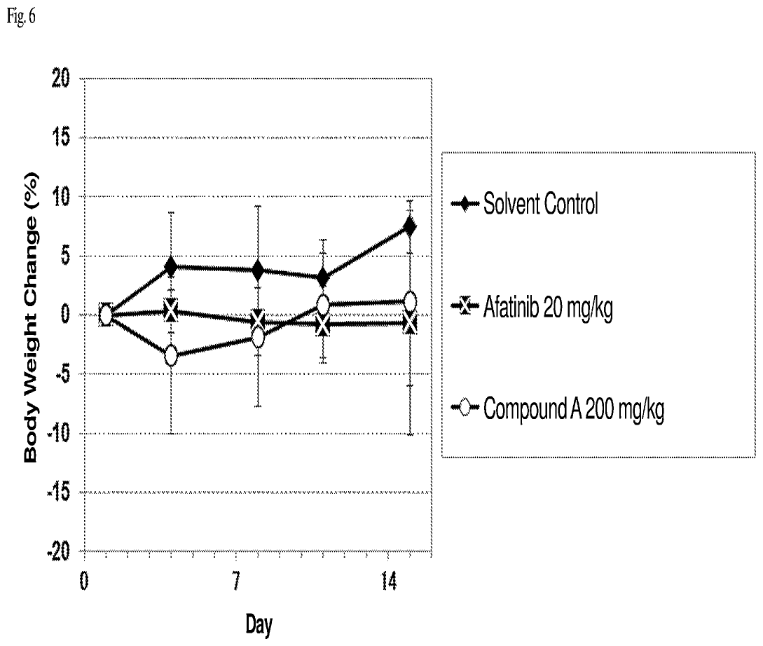

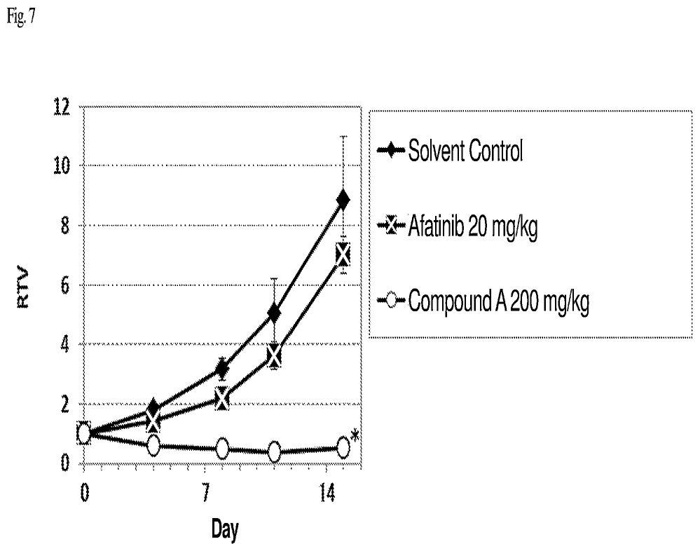

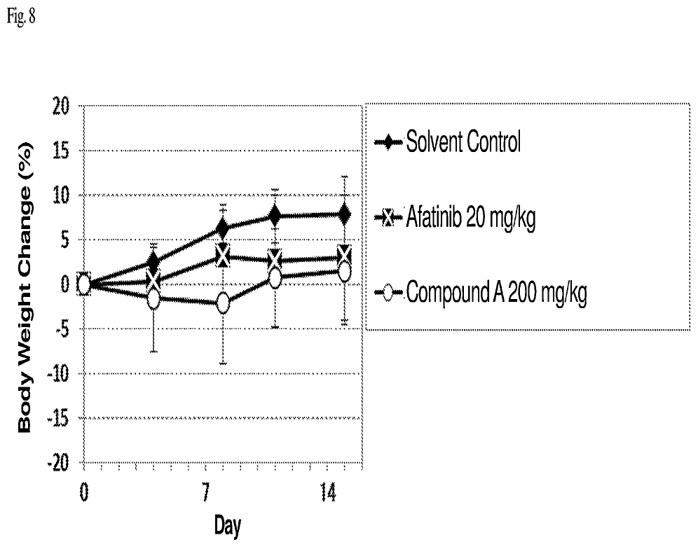

To compare the changes in growth of tumor over time due to administration of the individual test compounds, a relative tumor volume (which hereinafter also referred to as “RTV”) was calculated based on the tumor volume at the time the mice were divided into groups (which is taken as 1 for the tumor growth ratio), using the following formula. For a toxicity index, the body weight was measured over time, and the average body weight change (which hereinafter also referred to as “BWC (%)”) from the day on which the mice were divided into groups was calculated in accordance with the following formula. FIGS. 3 to 8 illustrate changes in the average RTV and the average BWC of the mice.

RTV=(the tumor volume on the day a tumor volume was measured)/(the tumor volume on the day mice were divided into groups)

BWC (%)=(the body weight measured on body weight measurement day)/(the body weight on the day mice were divided into groups)

When the average RTV of the group administered with compound A on the final evaluation day was smaller than the average RTV of the group administered with afatinib, while also exhibiting a statistically significant difference (Student's t-test, p<0.05), compound A was determined to be significantly more effective than afatinib. Such a case is indicated by the symbol “*” in the figures. The T/C (%) on the final evaluation day was calculated in accordance with the following formula. Table 4 illustrates the results.

TABLE 4

| Transplanted | | | |
|---|---|---|---|
| Tumor | Compound | 20 mg/kg | 200 mg/kg |
| NIH3T3 | Compound A of the | N.D. | 3 |
| EGFRinsASV | Present Invention | | |
| | Afatinib | 46 | N.D. |
| NIH3T3 | Compound A of the | N.D. | 5 |
| EGFRinsSVD | Present Invention | | |
| | Afatinib | 39 | N.D. |
| H1975 | Compound A of the | N.D. | 6 |
| EGFRinsSVD | Present Invention | | |
| | Afatinib | 79 | N.D. |

N.D.: No data are available.

As is clear from the results of FIGS. 3 to 8 and Table 4, compound A exhibited a remarkable antitumor effect on cell lines expressing EGFR exon 20 insertion mutation subcutaneously transplanted into nude mice. The effect was also higher than that of afatinib, without symptoms such as serious weight loss, abnormal feces, or abnormal skin in mice.

### Test Example 6

### In Vivo Drug Efficacy Test

Evaluation of Antitumor Effect on Model Subcutaneously Transplanted with Mutant EGFR-Expressing Cell Lines

Nude mice were subcutaneously transplanted with NIH3T3-EGFRinsNPH cells into which human mutant EGFR was introduced. At the point at which the tumor volume of the tumor engrafted in the nude mice grew to about 100 to 200 mm³, the mice were allocated into groups, 6 mice for each group, by stratified randomization such that the average tumor volume between the groups was uniform. The mice were then orally administered compound A or afatinib once daily for 10 consecutive days.

The dose of afatinib was 20 mg/kg/day, which is the maximum tolerated dose (the highest dose at which the weight loss during a dosing period is less than 20%); and the dose of compound A was 100, and 200 mg/kg/day. The maximum tolerated dose was determined in accordance with the “Guidelines Involving Experimental Neoplasia Proposals in Mice and Rats” of the National Cancer Institute (NCI), from a humanitarian perspective.

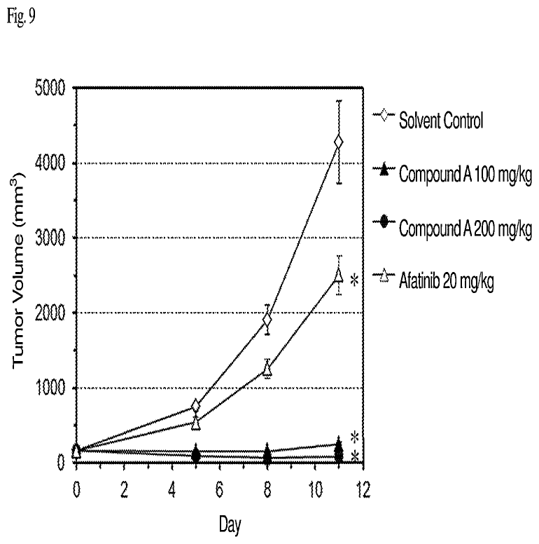

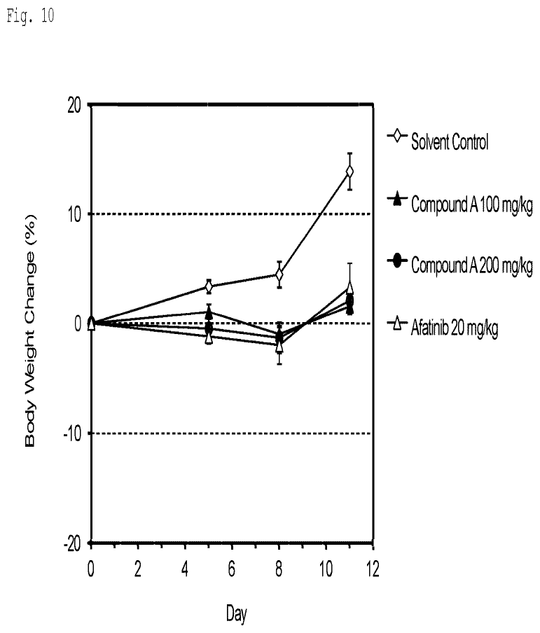

To compare the changes in growth of tumor over time due to administration of the individual test compounds, the tumor volume (which hereinafter also referred to as “TV”) of each mouse was calculated using the following formula. For a toxicity index, the body weight was measured over time, and the body weight change (which hereinafter also referred to as “BWC (%)”) from the day on which the mice were divided into groups was calculated in accordance with the following formula. FIGS. 9 and 10 illustrate changes in the average TV and the average BWC of the mice.

TV (mm³)=(the major axis×the short axis²)/2

BWC (%)=(the body weight measured on body weight measurement day)/(the body weight on the day mice were divided into groups)

When the average TV of the group administered with compound A on the day following the final administration was smaller than the average TV of the control group, while also exhibiting a statistically significant difference (Dunnett's test, p<0.05), compound A was determined to be effective in antitumor effect. Such a case is indicated by the symbol “*” in the figures. The T/C (%) on the final evaluation day was calculated in accordance with the following formula. Table 5 illustrates the results.

T/C (%)=(the tumor volume of the group administered with a test compound)/(the tumor volume of the control group)

TABLE 5

| Transplanted | | | |
|---|---|---|---|
| Tumor | Compound | 20 mg/kg | 200 mg/kg |
| NIH3T3 | Compound A | N.D. | 2 |
| EGFRinsNPH | Afatinib | 59 | N.D. |

N.D.: No data are available.

As is clear from FIGS. 9 and 10, and Table 5, compound A of the present invention exhibited a remarkable antitumor effect on cell lines expressing EGFR exon 20 insertion mutation subcutaneously transplanted into nude mice, accompanied by tumor growth inhibition or regression of tumor. In the evaluation, the mice also did not show serious weight loss.

### Test Example 7

Evaluation of Antitumor Effect on Rat Model Subcutaneously Transplanted with Mutant EGFR-Expressing Cell Lines

Nude rats were subcutaneously transplanted with H1975-EGFRinsSVD cells into which human mutant EGFR was introduced. At the point at which the tumor volume of the tumor engrafted in the nude rats grew to about 200 to 500 mm³, the rats were allocated into groups, 6 rats for each group, by stratified randomization such that the average tumor volume between the groups was uniform. The rats were then orally administered compound A once daily for 14 consecutive days.

The dose was 20 or 40 mg/kg/day, which is less than the maximum tolerated dose (the highest dose at which the weight loss during a dosing period is less than 20%) for 14 days, a dosing period of this test. The maximum tolerated dose was determined in accordance with the “Guidelines Involving Experimental Neoplasia Proposals in Mice and Rats” of the National Cancer Institute (NCI), from a humanitarian perspective.

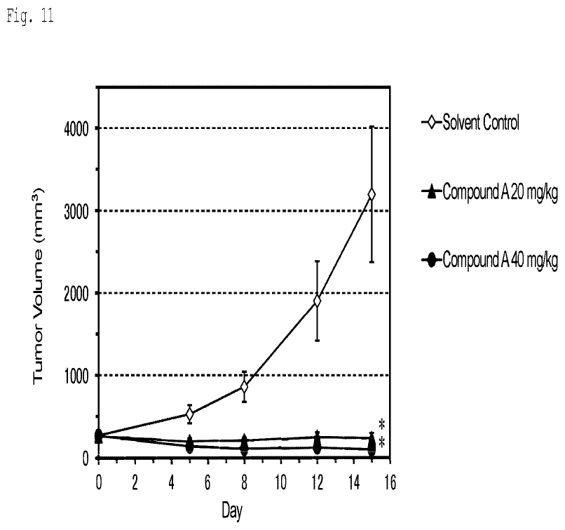

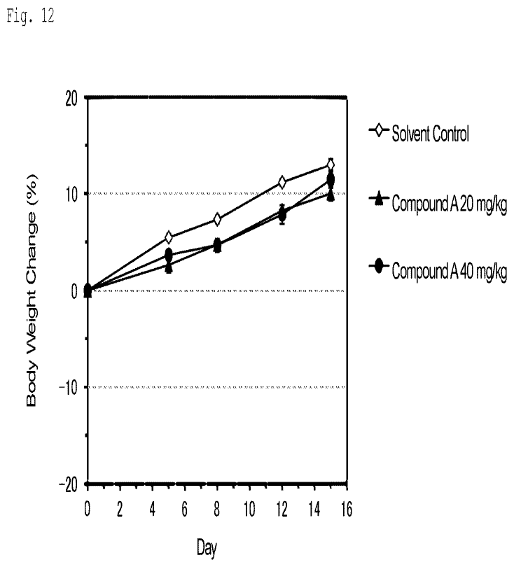

To compare the changes in growth of tumor over time due to administration of the test compound, the tumor volume (which hereinafter also referred to as “TV”) of each rat was calculated using the following formula. For a toxicity index, the body weight was measured over time, and the body weight change (which hereinafter also referred to as “BWC (%)”) from the day on which the rats were divided into groups was calculated in accordance with the following formula. FIGS. 11 and 12 illustrate changes in the average TV and the average BWC of the rats.

TV (mm³)=(the major axis×the short axis²)/2

BWC (%)=(the body weight measured on body weight measurement day)/(the body weight on the day rats were divided into groups)

When the average TV of the group administered with compound A on the final evaluation day was smaller than the average TV of the control group, while also exhibiting a statistically significant difference (Dunnett' s test, p<0.05), compound A was determined to be effective in antitumor effect. Such a case is indicated by the symbol “*” in the figures. The T/C (%) on the final evaluation day was calculated in accordance with the following formula. Table 6 illustrates the results.

T/C (%)=(the tumor volume of the group administered with a test compound)/(the tumor volume of the control group)

TABLE 6

| Transplanted | | | |
|---|---|---|---|
| Tumor | Compound | 20 mg/kg | 40 mg/kg |
| H1975 | Compound A | 7 | 3 |
| EGFRinsSVD | | | |

As is clear from FIGS. 11 and 12, and Table 6, compound A exhibited a remarkable antitumor effect on cell lines expressing EGFR exon 20 insertion mutation subcutaneously transplanted into nude rats, accompanied by tumor growth inhibition or regression of tumor. In the evaluation, the rats did not show serious weight loss.

### Test Example 8

Evaluation of Antitumor Effect on Mouse Model Subcutaneously Transplanted with Tumor Derived from Mutant EGFR-Positive Lung Cancer Patient

Nude mice were subcutaneously transplanted with LXF 2478, which is a tumor derived from a human lung cancer patient who was positive for EGFR with mutation V769_D770insASV. At the point at which the tumor volume of the tumor engrafted in the nude mice grew to about 100 to 200 mm³, the mice were allocated into groups, 8 mice for each group, by stratified randomization such that the average tumor volume between the groups was uniform. The mice were then orally administered compound A or afatinib once daily for 28 consecutive days, and a two-week observation period was set.

The dose of afatinib was 20 mg/kg/day, which is the maximum tolerated dose (the highest dose at which the weight loss during a dosing period is less than 20%); and the dose of compound A was 100, and 200 mg/kg/day. The maximum tolerated dose was determined in accordance with the “Guidelines Involving Experimental Neoplasia Proposals in Mice and Rats” of the National Cancer Institute (NCI), from a humanitarian perspective.

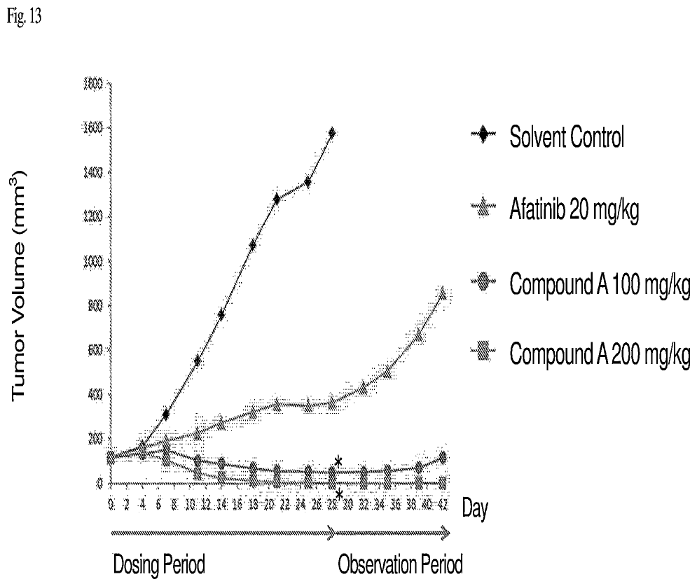

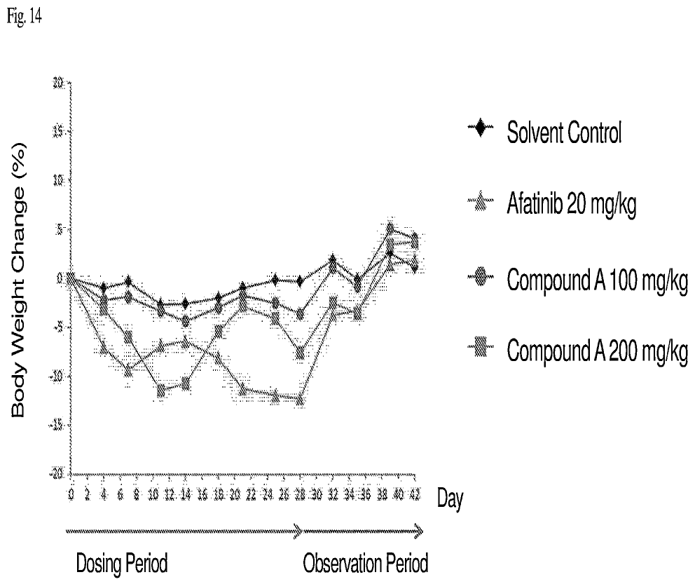

To compare the changes in growth of tumor over time due to administration of the individual test compounds, a relative tumor volume (which hereinafter also referred to as “RTV”) was calculated based on the tumor volume at the time the mice were divided into groups (which is taken as 1 for the tumor growth ratio) using the following formula. For a toxicity index, the body weight was measured over time, and the body weight change (which hereinafter also referred to as “BWC (%)”) from the day on which the mice were divided into groups was calculated in accordance with the following formula. FIGS. 13 and 14 illustrate changes in the average RTV and the average BWC of the mice.

RTV=(the tumor volume on the day a tumor volume was measured)/(the tumor volume on the day mice were divided into groups)

BWC (%)=(the body weight measured on body weight measurement day)/(the body weight on the day mice were divided into groups)

When the average RTV of the group administered with compound A on the day following the final administration (day 28) was smaller than the average RTV of the control group, while also exhibiting a statistically significant difference (Dunnett's test, p<0.05), compound A was determined to be effective. Such a case is indicated by the symbol “*” in the figures. The T/C (%) on the day following the final administration (day 28) was calculated in accordance with the following formula. Table 7 illustrates the results.

T/C (%)=(RTV of the group administered with a test compound)/(RTV of the control group)

TABLE 7

| Transplanted | | | | |
|---|---|---|---|---|
| Tumor | Compound | 20 mg/kg | 100 mg/kg | 200 mg/kg |
| LXF2478 | Compound A | N.D. | 2.0 | 0.1 |
| | Afatinib | 16.8 | N.D. | N.D. |

As is clear from FIGS. 13 and 14, and Table 7, compound A exhibited a remarkable antitumor effect on the tumor derived from a lung cancer patient who was positive for EGFR exon 20 insertion mutation subcutaneously transplanted into nude mice, accompanied by regression of tumor. The effect persisted over the observation period, and the mice did not show serious weight loss.

### Test Example 9

Evaluation of Life-Extending Effect on Model Transplanted with Mutant EGFR-Expressing Cell Lines in Lung

H1975-EGFRinsSVD-Luc strain was established by introducing a luciferase into H1975-EGFRinsSVD, which is a human mutant EGFR-introduced cell line. A pJTI (trademark) Fast DEST vector, which was prepared by encoding a Luciferase into NCI-H1975-EGFRinsSVD cells, was introduced into H1975-EGFRinsSVD-Luc cells, together with a pJTI (trademark) PhiC31 integrase expression vector, by electroporation using an Amaxa (trademark) Cell Line Nucleofector (trademark) Kit R, followed by selection using hygromycin B (Nacalai Tesque Inc.).

In evaluation of the life-extending effect, an equivalent amount of Matrigel was added to a suspension of cultured H1975-EGFRinsSVD-Luc cells to prepare a cell suspension, and the cell suspension was transplanted into the right lung of nude mice. On day 6 after transplantation, all of the living mice were administered a luciferin through the tail vein, and allocated into groups, 9 mice for each group, by stratified randomization such that the average luminescence intensity between the groups was uniform. The mice were then orally administered compound A or afatinib once daily on consecutive days. The dose of afatinib was 20 mg/kg/day, which is the maximum tolerated dose (the highest dose at which the weight loss during a dosing period is less than 20%); and the dose of compound A was 100, and 200 mg/kg/day. The maximum tolerated dose was determined in accordance with the “Guidelines Involving Experimental Neoplasia Proposals in Mice and Rats” of the National Cancer Institute (NCI), from a humanitarian perspective.

To evaluate the life-extending effect, the survival period after transplantation was observed, and the survival time of each mouse was determined. From the survival time, the median survival time (which hereinafter also referred to as “MST”) of each group was calculated, and the survival period-extending effect (i.e., an increase in lifespan, which hereinafter also referred to as “I.L.S. (%)”) was calculated based on MST of the control group and the group administered with a test compound, using the following formula. For a toxicity index, the body weight was measured over time, and the body weight change (which hereinafter also referred to as “BWC (%)”) from the day on which the mice were divided into groups was calculated in accordance with the following formula.

I.L.S. (%)=(T/C-1)×100
T: MST of the group administered with a test compound
C: MST of the control group

BWC (%)=(the body weight measured on body weight measurement day)/(the body weight on the day mice were divided into groups)

When the MST of the group administered with compound A was larger than the MST of the control group, while exhibiting a statistically significant difference (Wilcoxon test, p<0.05), compound A was determined to be effective in a life-extending effect. Table 8 illustrates the results.

TABLE 8

| Transplanted | | | | |
|---|---|---|---|---|
| Tumor | Compound | MST | I.L.S. (%) | p value |
| H1975 | Solvent Control | 44 | N.A. | N.A. |
| EGFR | Compound A | 70 | 59 | <0.01 |
| insSVD | 100 mg/kg | | | |
| | Compound A | 89 | 102 | <0.01 |
| | 200 mg/kg | | | |
| | Afatinib | 54 | 23 | N.S. |
| | 20 mg/kg | | | |

N.A.: Analysis was not applicable.
N.S.: No significant difference was observed.

As is clear from Table 8, compound A exhibited a remarkable life-extending effect on the nude mouse models transplanted in the same part of their lung with cell lines expressing EGFR exon 20 insertion mutation. However, afatinib did not exhibit such a life-extending effect on the mouse models. The mice administered with compound A also did not show serious weight loss.

### Test Example 10

### Evaluation of Phosphorylated-EGFR Inhibitory Activity in Transplanted Tumor and Mouse Skin Tissue

Nude mice were subcutaneously transplanted with NIH3T3-EGFRinsSVD cells into which human mutant EGFR was introduced. At the point at which the tumor volume of the tumor engrafted in the nude mice grew to about 250 to 500 mm³, the mice were allocated into groups, 3 mice for each group, by stratified randomization such that the average tumor volume between the groups was uniform. The mice were then orally administered compound A or afatinib once. One hour and three hours after administration, which are respectively around the time at which the maximum blood concentration of compound A and afatinib is achieved, their tumor and skin tissue were collected. The collected tissue was subjected to flash-freezing with liquid nitrogen, and stored at −80° C. until use. The tumor and skin tissue were homogenized, with a RIPA buffer (Thermo Fisher Scientific) containing a protease inhibitor cocktail (Thermo Fisher Scientific) added, and proteins within the cells were extracted. The protein concentration was measured with a BCA protein assay kit (Thermo Fisher Scientific), and each sample was adjusted to a protein concentration suitable for measurement of phosphorylated EGFR expression. The proteins were separated by SDS-PAGE, and transferred onto a PVDF membrane. After blocking, Phospho-EGF Receptor (Tyr1068) #2234 (CST), which is a primary antibody, was diluted with a 0.1% TBS-T buffer to 1/1000, and allowed to react at 4° C. overnight. Thereafter, an HRP-labeled anti-rabbit antibody #NA9340V (GE Healthcare), which is a secondary antibody, was diluted to 1/2500 with a 5% skim milk solution adjusted with a 0.1% TBS-T buffer, and allowed to react at room temperature for 40 minutes. After reaction with ECL-Prime (GE Healthcare), detection was performed with an LAS-3000 image analyzer (GE Healthcare).

The test results reveal that compound A selectively inhibits mutant EGFR in the tumor over wild-type EGFR in the skin.

### SEQUENCE LISTING

CLN-027US_SequenceListing.txt.

What is claimed is:

1. An antitumor agent for treating a malignant tumor patient expressing EGFR having exon 20 insertion mutation, the antitumor agent comprising a compound selected from the group consisting of:
(S)-N-(4-amino-6-methyl-5-(quinolin-3-yl)-8,9-dihydropyrimido[5,4-b]indolizin-8-yl)acrylamide;
(S)-N-(4-amino-6-methylene-5-(quinolin-3-yl)-7,8-dihydro-6H-pyrimido[5,4-b]pyrrolizin-7-yl)acrylamide;
(S,E)-N-(4-amino-6-methylene-5-(quinolin-3-yl)-7,8-dihydro-6H-pyrimido[5,4-b]pyrrolizin-7-yl)-3-chloroacrylamide; and
(R)-N-(4-amino-6-methyl-5-(quinolin-3-yl)-8,9-dihydropyrimido[5,4-b]indolizin-8-yl)-N-methylacrylamide,
or a salt thereof.

2. The antitumor agent according to claim 1, wherein the compound is (S)-N-(4-amino-6-methyl-5-(quinolin-3-yl)-8,9-dihydropyrimido[5,4-b]indolizin-8-yl)acrylamide.

3. The antitumor agent according to claim 1, wherein the malignant tumor patient expressing EGFR having exon 20 insertion mutation is a patient with lung cancer, breast cancer, head and neck cancer, brain tumor, uterine cancer, hematopoietic tumor, or skin cancer.

4. The antitumor agent according to claim 1, wherein the malignant tumor patient expressing EGFR having exon 20 insertion mutation is a lung cancer patient.

5. The antitumor agent according to claim 1, wherein the exon 20 insertion mutation is a mutation in which one or more amino acids are inserted in the exon 20 region.

6. The antitumor agent according to claim 1, wherein the exon 20 insertion mutation is a mutation in which 1 to 7 amino acids are inserted in the exon 20 region.

7. The antitumor agent according to claim 1, wherein the exon 20 insertion mutation is a mutation in which 1 to 4 amino acids are inserted in the exon 20 region.

8. The antitumor agent according to claim 1, wherein the exon 20 insertion mutation is V769_D770insASV, D770_N771insSVD, D770_N771insG, H773_V774insNPH, or H773_V774insPH.

9. A method for treating a malignant tumor patient, comprising the step of administering an effective amount of a compound selected from the group consisting of:
(S)-N-(4-amino-6-methyl-5-(quinolin-3-yl)-8,9-dihydropyrimido[5,4-b]indolizin-8-yl)acrylamide;
(S)-N-(4-amino-6-methylene-5-(quinolin-3-yl)-7,8-dihydro-6H-pyrimido[5,4-b]pyrrolizin-7-yl)acrylamide;
(S,E)-N-(4-amino-6-methylene-5-(quinolin-3-yl)-7,8-dihydro-6H-pyrimido[5,4-b]pyrrolizin-7-yl)-3-chloroacrylamide; and
(R)-N-(4-amino-6-methyl-5-(quinolin-3-yl)-8,9-dihydropyrimido[5,4-b]indolizin-8-yl)-N-methylacrylamide,
or a salt thereof, to a malignant tumor patient expressing EGFR having exon 20 insertion mutation.

10. A compound selected from the group consisting of:
(S)-N-(4-amino-6-methyl-5-(quinolin-3-yl)-8,9-dihydropyrimido[5,4-b]indolizin-8-yl)acrylamide;
(S)-N-(4-amino-6-methylene-5-(quinolin-3-yl)-7,8-dihydro-6H-pyrimido[5,4-b]pyrrolizin-7-yl)acrylamide;
(S,E)-N-(4-amino-6-methylene-5-(quinolin-3-yl)-7,8-dihydro-6H-pyrimido[5,4-b]pyrrolizin-7-yl)-3-chloroacrylamide; and
(R)-N-(4-amino-6-methyl-5-(quinolin-3-yl)-8,9-dihydropyrimido[5,4-b]indolizin-8-yl)-N-methylacrylamide,
or a salt thereof, to treat a malignant tumor patient expressing EGFR having exon 20 insertion mutation.

11. Use of a compound selected from the group consisting of:
(S)-N-(4-amino-6-methyl-5-(quinolin-3-yl)-8,9-dihydropyrimido[5,4-b]indolizin-8-yl)acrylamide;
(S)-N-(4-amino-6-methylene-5-(quinolin-3-yl)-7,8-dihydro-6H-pyrimido[5,4-b]pyrrolizin-7-yl)acrylamide; and
(S,E)-N-(4-amino-6-methylene-5-(quinolin-3-yl)-7,8-dihydro-6H-pyrimido[5,4-b]pyrrolizin-7-yl)-3-chloroacrylamide; and
(R)-N-(4-amino-6-methyl-5-(quinolin-3-yl)-8,9-dihydropyrimido[5,4-b]indolizin-8-yl)-N-methylacrylamide,
or a salt thereof for the production of a pharmaceutical agent for treating a malignant tumor patient expressing EGFR having exon 20 insertion mutation.


SEQUENCE LISTING (1 sequence)

SEQ ID NO: 1 - protein, 1210 aa, Homo sapiens [sequence omitted]